(19) United States
(12) Patent Application Publication
(10) Pub. No.: US 2025/0186022 A1
(43) Pub. Date: Jun. 12, 2025

(54) ULTRASOUND DIAGNOSTIC APPARATUS

(71) Applicant: FUJIFILM Corporation, Tokyo (JP)

(72) Inventors: Takehiro TSUJITA, Tokyo (JP); Tsuyoshi MATSUMOTO, Tokyo (JP)

(73) Assignee: FUJIFILM Corporation, Tokyo (JP)

(21) Appl. No.: 18/966,036

(22) Filed: Dec. 2, 2024

(30) Foreign Application Priority Data
Dec. 7, 2023 (JP) 2023-207124

(51) Int. Cl. A61B 8/00; G16H 10/60; G16H 30/40

(52) U.S. Cl. CPC A61B 8/4254; A61B 8/461; G16H 10/60; G16H 30/40

(57) ABSTRACT

An ultrasound diagnostic apparatus including a position sensor, a recognition unit, a transformation unit, a display controller, and an update unit is provided. The position sensor acquires position information of an ultrasound probe. The recognition unit recognizes, based on an ultrasound image acquired by scanning with an ultrasonic wave using the ultrasound probe, a scanning cross section scanned with the ultrasonic wave. The transformation unit uses an anatomical model in which a correct answer cross section on which ultrasonic examination is to be performed and a correct answer position of the correct answer cross section are registered, to transform a position indicated by the position information into the correct answer position associated with the anatomical model according to a transformation rule. The display controller displays extraction information related to the ultrasonic examination, which corresponds to the correct answer position, on a display unit. The update unit updates the transformation rule by using the correct answer position of the correct answer cross section corresponding to the scanning cross section recognized by the recognition unit and the position indicated by the position information.

### CROSS-REFERENCE TO RELATED APPLICATION

This application claims priority under 35 USC 119 from Japanese Patent Application No. 2023-207124, filed 7 Dec. 2023, the disclosure of which is incorporated by reference herein.

### BACKGROUND OF THE INVENTION

### 1. Field of the Invention

The present disclosure relates to an ultrasound diagnostic apparatus.

### 2. Description of the Related Art

In ultrasonic examination, a user such as an examination technician may specify a scanning position of an ultrasonic wave while viewing an ultrasound image.

US2008/0154123A discloses a technique of specifying a cross section scanned with an ultrasonic wave by combining a position detected by a position sensor provided in an ultrasound probe and image analysis.

JP2010-131269A discloses a technique of specifying a cross section scanned by an ultrasonic wave by using a computed tomography (CT) image and an ultrasound image.

### SUMMARY OF THE INVENTION

In general, experience or knowledge is required for a user to accurately specify a scanning position. Therefore, it is not easy for the user to specify the scanning position. In addition, since the specification of the scanning position depends on the experience or knowledge of the user, the specified scanning position lacks objectivity.

An object of the present disclosure is to reflect a scanning position of an ultrasonic wave in a subject-independent model using a simple method.

One aspect of the present disclosure relates to an ultrasound diagnostic apparatus comprising: an acquisition unit that acquires position information of an ultrasound probe; a recognition unit that recognizes, based on an ultrasound image acquired by scanning with an ultrasonic wave using the ultrasound probe, a scanning cross section scanned with the ultrasonic wave; a transformation unit that uses an anatomical model in which a correct answer cross section on which ultrasonic examination is to be performed and a correct answer position of the correct answer cross section are registered, to transform a position indicated by the position information acquired by the acquisition unit into the correct answer position associated with the anatomical model according to a transformation rule; a display controller that displays extraction information related to the ultrasonic examination, which corresponds to the correct answer position transformed by the transformation unit, on a display; and an update unit that updates the transformation rule by using the correct answer position of the correct answer cross section corresponding to the scanning cross section recognized by the recognition unit and the position indicated by the position information acquired by the acquisition unit.

The transformation according to the transformation rule may be transformation using a transformation matrix, and the update unit may update the transformation matrix by using the position indicated by the position information acquired by the acquisition unit and the correct answer position of the correct answer cross section.

The correct answer cross section and the correct answer position may be registered in the anatomical model for each examination site.

The ultrasound diagnostic apparatus may further comprise: a setting unit that sets an anatomical model corresponding to at least one of a body shape or a body position of a subject that serves as a target of the ultrasonic examination, as the anatomical model to be used for the transformation.

At least one of the body shape or the body position of the subject may be designated by a user, and the setting unit may set an anatomical model corresponding to the at least one designated by the user, as the anatomical model to be used for the transformation.

The setting unit may estimate at least one of the body shape or the body position of the subject based on an image generated by imaging the subject with a camera, and set an anatomical model corresponding to a result of the estimation, as the anatomical model to be used for the transformation.

The setting unit may receive subject identification information for identifying the subject, and set an anatomical model corresponding to a body shape indicated by body shape information linked to the subject identification information, as the anatomical model to be used for the transformation.

The ultrasound diagnostic apparatus may further comprise: an estimation unit that estimates at least one of the body shape or the body position of the subject based on a position of the ultrasound probe, and the setting unit may set an anatomical model corresponding to a result of the estimation by the estimation unit, as the anatomical model to be used for the transformation.

The ultrasound diagnostic apparatus may further comprise: an estimation unit that estimates at least one of the body shape or the body position of the subject based on the ultrasound image, and the setting unit may set an anatomical model corresponding to a result of the estimation by the estimation unit, as the anatomical model to be used for the transformation.

In a case in which a change occurs in the body position of the subject, the update unit may create a new transformation rule corresponding to the changed body position.

The extraction information may include at least one of information indicating a position of the ultrasound probe, information indicating an orientation of the ultrasound probe, information indicating a current scanning range with an ultrasonic wave, information indicating a past scanning range with an ultrasonic wave, or information indicating a non-scanned range.

According to the present disclosure, it is possible to reflect a scanning position of an ultrasonic wave in a subject-independent model using a simple method.

### BRIEF DESCRIPTION OF THE DRAWINGS

FIG. 1 is a block diagram showing an example of a configuration of an ultrasound diagnostic apparatus according to an embodiment.

FIG. 2 is a diagram showing an anatomical model.

FIG. 3 is a diagram showing a subject.

FIG. 4 is a diagram showing a display example of an ultrasound image and a body mark.

FIG. 5 is a diagram showing a display example of an ultrasound image and a body mark.

FIG. 6 is a diagram showing a display example of an ultrasound image and a body mark.

FIG. 7 is a diagram showing a display example of an ultrasound image and a body mark.

FIG. 8 is a diagram showing a display example of an ultrasound image.

### DESCRIPTION OF THE PREFERRED EMBODIMENTS

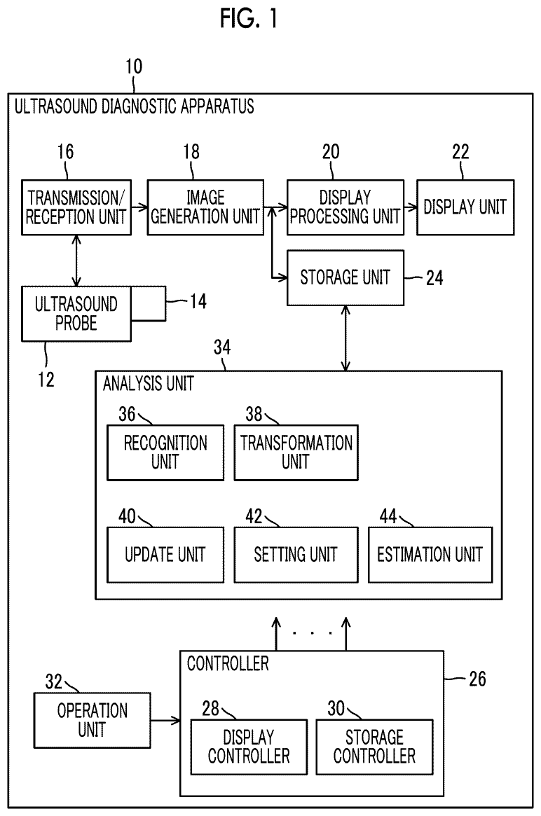

An ultrasound diagnostic apparatus 10 according to an embodiment will be described with reference to FIG. 1. FIG. 1 is a block diagram showing an example of a configuration of the ultrasound diagnostic apparatus 10 according to the embodiment.

The ultrasound diagnostic apparatus 10 generates ultrasound image data by transmitting and receiving ultrasonic waves using an ultrasound probe 12. For example, the ultrasound diagnostic apparatus 10 transmits the ultrasonic waves into a subject and receives the ultrasonic waves reflected from an inside of the subject to generate ultrasound image data representing a tissue inside the subject.

The ultrasound probe 12 is a device that transmits and receives the ultrasonic waves. The ultrasound probe 12 includes, for example, a 1D array oscillator. The 1D array oscillator includes a plurality of ultrasound oscillators arranged one-dimensionally. An ultrasonic beam is formed by the 1D array oscillator, and electron scanning with the ultrasonic beam is repeatedly performed. As a result, a scanning cross section is formed in a living body for each electron scanning. The scanning cross section corresponds to a two-dimensional echo data acquisition space. The ultrasound probe 12 may include a 2D array oscillator formed by two-dimensionally arranging a plurality of ultrasound oscillators. In a case in which the ultrasonic beam is formed by the 2D array oscillator and the electron scanning with the ultrasonic beam is repeatedly performed, the scanning cross section as the two-dimensional echo data acquisition space is formed for each electron scanning. In a case in which the scanning with the ultrasonic beam is performed two-dimensionally, a three-dimensional space as a three-dimensional echo data acquisition space is formed. As the scanning method, sector scanning, linear scanning, convex scanning, or the like is used.

The position sensor 14 is provided in the ultrasound probe 12 and detects a position of the ultrasound probe 12. Position information indicating the position of the ultrasound probe 12 is output to an analysis unit 34. Hereinafter, the position of the ultrasound probe 12 detected by the position sensor 14 is referred to as an “actual position”. The actual position is a position at which the ultrasound probe 12 is actually provided. For example, the position sensor 14 detects a relative position, a rotation direction, a rotation speed, and a tilt (for example, a posture) of the ultrasound probe 12 with respect to the subject. The position information includes information indicating the relative position, information indicating the rotation direction, information indicating the rotation speed, and information indicating the tilt. The position sensor 14 corresponds to an example of an acquisition unit.

A known sensor is used as the position sensor 14. For example, the position sensor 14 is a sensor (for example, an acceleration sensor and a gyro sensor) that detects a three-axis acceleration and a three-axis angular velocity. The position sensor 14 may be a magnetic sensor. In a case in which the magnetic sensor is used as the position sensor 14, the magnetic sensor detects the actual position of the ultrasound probe 12 by measuring an intensity of a magnetic field emitted from a magnetic field generator installed at a predetermined position.

A transmission/reception unit 16 functions as a transmission beam former and a reception beam former. In the transmission, the transmission/reception unit 16 supplies a plurality of transmission signals having a certain delay relationship to the plurality of ultrasound oscillators included in the ultrasound probe 12. As a result, a transmission beam of the ultrasonic waves is formed. In the reception, a reflected wave (RF signal) from the living body is received by the ultrasound probe 12, whereby a plurality of reception signals are output from the ultrasound probe 12 to the transmission/reception unit 16. The transmission/reception unit 16 forms a reception beam by applying phasing addition processing to the plurality of reception signals. Data of the reception beam is output to an image generation unit 18. That is, the transmission/reception unit 16 forms the reception beam by performing delay processing on the reception signal obtained from each ultrasound oscillator in accordance with a delay processing condition for each ultrasound oscillator and performing addition processing on the plurality of reception signals obtained from the plurality of ultrasound oscillators. The delay processing condition is defined by reception delay data indicating a delay time. A reception delay data set (that is, a set of delay times) corresponding to the plurality of ultrasound oscillators is supplied from a controller 26.

The electron scanning with the ultrasonic beam (that is, the transmission beam and the reception beam) is performed by the action of the transmission/reception unit 16, thereby forming a scanning cross section. The scanning cross section corresponds to a plurality of beams, and the plurality of beams constitute a reception frame (specifically, an RF signal frame). Each beam is composed of a plurality of echoes arranged in a depth direction. By repeating the electron scanning with the ultrasonic beam, a plurality of reception frames arranged on a time axis are output from the transmission/reception unit 16 to the image generation unit 18. The plurality of reception frames constitute a reception frame sequence.

In a case in which the electron scanning with the ultrasonic beam is performed two-dimensionally by the action of the transmission/reception unit 16, the three-dimensional echo data acquisition space is formed, and volume data as an echo data aggregate is acquired from the three-dimensional echo data acquisition space. By repeating the electron scanning with the ultrasonic beam, a plurality of volume data arranged on a time axis are output from the transmission/reception unit 16 to the image generation unit 18. The plurality of volume data constitute a volume data sequence.

The image generation unit 18 generates ultrasound image data (for example, B-mode image data) by applying, to the reception frame output from the transmission/reception unit 16, signal processing such as detection, amplitude compression (for example, logarithmic compression), and a conversion function (a coordinate transformation function and an interpolation processing function by a digital scan converter (DSC)). Hereinafter, the image data will be appropriately referred to as an “image”. For example, the ultrasound image data will be appropriately referred to as an “ultrasound image”, and the B-mode image data will be appropriately referred to as a “B-mode image”. The ultrasound image according to the present embodiment is not limited to the B-mode image, and may be any image generated by the ultrasound diagnostic apparatus 10. For example, the ultrasound image according to the present embodiment may be a color Doppler image, a pulse Doppler image, a strain image, a shear wave elastography image, or the like.

A display processing unit 20 generates a display image by overlaying necessary graphic data on the ultrasound image. The display image is output to a display unit 22. One or a plurality of images are arranged and displayed in a display aspect according to a display mode.

The display unit 22 is a display such as a liquid crystal display or an EL display. The ultrasound image such as the B-mode image is displayed on the display unit 22. The display unit 22 may be a device comprising both a display and an operation unit 32. For example, a graphic user interface (GUI) may be implemented by the display unit 22. In addition, a user interface such as a touch panel may be implemented by the display unit 22.

A storage unit 24 constitutes one or a plurality of storage regions for storing data. The storage unit 24 is, for example, a hard disk drive (HDD), a solid state drive (SSD), various memories (for example, RAM, DRAM, or ROM), other storage devices (for example, optical disk), or a combination thereof.

The ultrasound image generated by the imaging of the ultrasound diagnostic apparatus 10, information indicating imaging conditions, information regarding the subject (for example, a patient), and the like are stored in the storage unit 24.

In addition, data of an anatomical model is stored in the storage unit 24 in advance. The anatomical model is a three-dimensional model in which a standard human body (for example, an upper body of a person) is imitated. The anatomical model is a standard phantom that is independent of the individual subject.

Correct answer cross section information indicating a cross section on which ultrasonic examination is to be performed (hereinafter, referred to as a “correct answer cross section”) and correct answer position information indicating a position of the correct answer cross section (hereinafter, referred to as a “correct answer position”) are registered in the anatomical model in advance. The correct answer cross section is a cross section to be scanned with the ultrasonic waves in the ultrasonic examination. The correct answer cross section information is information indicating a range of the correct answer cross section (that is, a two-dimensional range) and an orientation of the correct answer cross section. The correct answer position is a position on a three-dimensional model in which the human body is imitated (for example, a position on a body surface of the human body). For example, a plurality of different correct answer cross sections and the correct answer position of each correct answer cross section are registered in the anatomical model.

In the ultrasonic examination, a plurality of different correct answer cross sections on which the ultrasonic examination is performed are determined in advance, and a user such as an examination technician executes the ultrasonic examination by changing the position or the orientation of the ultrasound probe 12 on the subject such that each correct answer cross section is scanned with the ultrasonic waves.

The controller 26 controls an operation of each unit of the ultrasound diagnostic apparatus 10. The controller 26 includes a display controller 28 and a storage controller 30.

The display controller 28 displays the ultrasound image and other information on the display unit 22.

For example, the display controller 28 displays extraction information corresponding to the correct answer position on the display unit 22. The extraction information is information related to the ultrasonic examination. For example, the extraction information includes at least one of information indicating the current actual position of the ultrasound probe 12, information indicating the current orientation of the ultrasound probe 12, information indicating the current scanning range with the ultrasonic waves (for example, information indicating the current scanning cross section), information indicating the past scanning range with the ultrasonic waves (for example, information indicating the past scanning cross section), information indicating the non-scanned range (for example, information indicating the scanning cross section that has not yet been scanned with the ultrasonic waves), or information indicating the orientation of the viewpoint. The information included in the extraction information may be designated by the user or may be determined in advance. The information indicating the non-scanned range is information indicating the correct answer cross section.

For example, the information indicating the current scanning cross section is correct answer cross section information indicating a correct answer cross section corresponding to the current scanning cross section (that is, a correct answer cross section estimated to match the scanning cross section). The information indicating the past scanning cross section is correct answer cross section information indicating a correct answer cross section corresponding to the past scanning cross section. The information indicating the non-scanned scanning cross section is correct answer cross section information indicating a correct answer cross section corresponding to the non-scanned scanning cross section. These types of correct answer cross section information are registered in advance in the anatomical model and are stored in the storage unit 24 in advance.

The storage controller 30 stores various types of information in the storage unit 24 and reads out various types of information from the storage unit 24.

The operation unit 32 is a device for the user to input a condition, a command, and the like necessary for imaging, to the ultrasound diagnostic apparatus 10. For example, the operation unit 32 is an operation panel, a switch, a button, a keyboard, a mouse, a track ball, or a joystick. The analysis unit 34 analyzes the ultrasound image and specifies the correct answer position on the anatomical model corresponding to the actual position based on an analysis result and the actual position of the ultrasound probe 12 (that is, the position detected by the position sensor 14). The display controller 28 displays the extraction information corresponding to the specified correct answer position, on the display unit 22.

The analysis unit 34 specifies the correct answer position corresponding to the actual position according to a transformation rule. For example, the transformation according to the transformation rule is transformation using a transformation matrix. The transformation matrix is used in a method of specifying the correct answer position corresponding to the actual position. The analysis unit 34 obtains a transformation matrix for transforming the actual position into the correct answer position, and specifies the correct answer position corresponding to the actual position by using the transformation matrix. Further, the analysis unit 34 updates the transformation matrix using the actual position and the correct answer position. In this way, the accuracy of the position transformation is increased as the ultrasonic examination progresses.

For example, the analysis unit 34 includes a recognition unit 36, a transformation unit 38, an update unit 40, a setting unit 42, and an estimation unit 44.

The recognition unit 36 recognizes the scanning cross section scanned with the ultrasonic waves, based on the ultrasound image. For example, the recognition unit 36 estimates the scanning cross section by executing the cross section recognition processing on the ultrasound image. Estimating the scanning cross section is to specify the correct answer cross section from the anatomical model. That is, the recognition unit 36 specifies the correct answer cross section that is estimated to match the scanning cross section actually scanned with the ultrasonic waves, from the anatomical model. In addition, the recognition unit 36 calculates a degree of certainty representing the certainty of the estimation. The degree of certainty is also called likelihood. The degree of certainty is an indicator indicating a degree of match between the estimated scanning cross section and the correct answer cross section. The recognition unit 36 may estimate a plurality of the scanning cross sections and calculate a degree of certainty of each scanning cross section.

The recognition unit 36 may estimate the scanning cross section currently being scanned by executing the cross section recognition processing on the currently acquired ultrasound image. That is, the recognition unit 36 may estimate the scanning cross section in real time.

As another example, the recognition unit 36 may estimate the scanning cross section by executing the cross section recognition processing on the ultrasound image that has already been acquired and stored in the storage unit 24 or an external device.

As the cross section recognition processing according to the present embodiment, known cross section recognition processing is used. For example, machine learning or artificial intelligence (AI) may be used in the cross section recognition processing. The type of the machine learning or artificial intelligence used is not limited, and any algorithm or model may be used. For example, a convolutional neural network (CNN), a recurrent neural network (RNN), generative adversarial networks (GAN), linear models, random forests, decision tree learning, a support vector machine (SVM), an ensemble classifier, or other algorithms are used. A pattern matching algorithm such as template matching or an algorithm that does not require learning such as a correlation coefficient or a similarity degree calculation may be used in the cross section recognition processing.

For example, the recognition unit 36 estimates the scanning cross section by executing the cross section recognition processing using machine learning on the ultrasound image, and calculates a degree of certainty representing the certainty of the estimation using the machine learning.

The recognition unit 36 may estimate the scanning cross section scanned with the ultrasonic waves by comparing the ultrasound image (for example, the B-mode image) generated by the transmission and reception of the ultrasonic waves with the images of the plurality of correct answer cross sections (for example, the B-mode images). In this case, a technique such as pattern matching may be used.

The transformation unit 38 uses the anatomical model to transform an actual position of the ultrasound probe 12 detected by the position sensor 14 (hereinafter, referred to as an “actual position Pr”) into a correct answer position registered in the anatomical model (hereinafter, referred to as a “correct answer position Pm”) according to the transformation rule. That is, the transformation unit 38 transforms the actual position Pr of the ultrasound probe 12 into the correct answer position Pm on the anatomical model. The correct answer position Pm is a position on a three-dimensional coordinate system.

The transformation of the actual position Pr into the correct answer position Pm corresponds to specifying which correct answer position Pm on the anatomical model the actual position Pr of the ultrasound probe 12 corresponds to. That is, the correct answer position Pm obtained by transforming the actual position Pr corresponds to the position of the ultrasound probe 12 on the anatomical model.

The transformation according to the transformation rule is transformation using the transformation matrix M. The actual position Pr and the correct answer position Pm of the ultrasound probe 12 are transformed into each other by using the transformation matrix M. For example, a relationship between the actual position Pr and the correct answer position Pm is expressed by Expression (1) by using the transformation matrix M.

$$Pm = M \cdot Pr \qquad ( 1 )$$

In a case in which the actual position Pr and the correct answer position Pm are expressed by a matrix, the transformation matrix M is expressed by Expression (2).

$$M = Pm \cdot {Pr}^{- 1} \qquad ( 2 )$$

The transformation unit 38 transforms the actual position Pr detected by the position sensor 14 into the correct answer position Pm on the anatomical model in accordance with Expression (1).

An initial transformation matrix M is determined in advance and is stored in the storage unit 24 in advance. As will be described below, the transformation matrix M is updated from time to time as the examination progresses. In a case in which the transformation matrix M is updated, the transformation unit 38 transforms the actual position Pr into the correct answer position Pm by using the updated transformation matrix M. The transformation matrix M is updated, thereby improving the accuracy of the transformation.

The update unit 40 updates the transformation rule (for example, the transformation matrix M) by using the correct answer position Pm of the correct answer cross section corresponding to the scanning cross section recognized by the recognition unit 36 and the actual position Pr of the ultrasound probe 12. Specifically, the update unit 40 updates the transformation matrix M in accordance with Expression (2). Hereinafter, the update of the transformation matrix M will be described in detail.

As described above, the actual position Pr of the ultrasound probe 12 is detected by the position sensor 14. The recognition unit 36 estimates the scanning cross section scanned with the ultrasonic waves. That is, the correct answer cross section estimated to match the scanning cross section actually scanned with the ultrasonic waves is specified from the anatomical model. The correct answer cross section and the correct answer position Pm are registered in the anatomical model, and the update unit 40 specifies the correct answer position Pm of the estimated correct answer cross section from the anatomical model. The update unit 40 calculates the transformation matrix M by inputting the actual position Pr and the correct answer position Pm to Expression (2).

For example, in a case in which the ultrasonic examination progresses and the scanning with the ultrasonic waves is performed at the plurality of different actual positions Pr, the plurality of different actual positions Pr are detected by the position sensor 14. In addition, for each actual position Pr, the correct answer cross section corresponding to the actual scanning cross section is specified, and the correct answer position of the correct answer cross section is specified. For example, the update unit 40 calculates the transformation matrix M in accordance with Expression (2) each time the actual position Pr and the correct answer position Pm are obtained. In this way, the update unit 40 updates the transformation matrix M.

The setting unit 42 sets the anatomical model and the transformation rule (for example, the transformation matrix M) for at least one of a body shape or a body position of the subject.

The estimation unit 44 estimates at least one of the body shape or the body position of the subject based on the position and the posture of the ultrasound probe 12. The setting unit 42 sets an anatomical model corresponding to a result of the estimation by the estimation unit 44 as the anatomical model to be used for the transformation.

Details of the setting unit 42 and the estimation unit 44 will be described below. The setting unit 42 and the estimation unit 44 do not necessarily have to be provided in the ultrasound diagnostic apparatus 10.

Hereinafter, the present embodiment will be described in more detail.

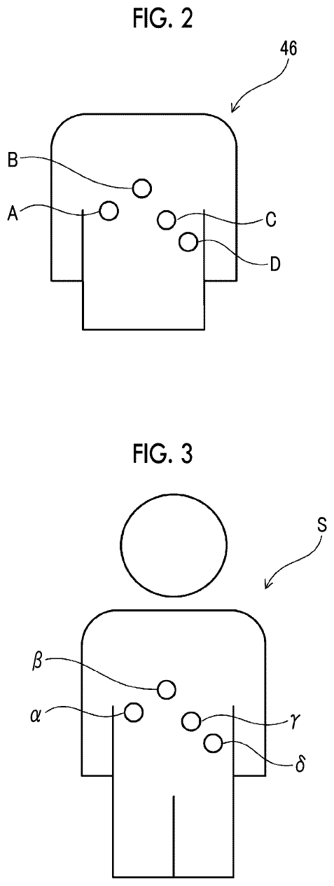

The update by the update unit 40 will be described with reference to FIGS. 2 and 3. FIG. 2 shows an anatomical model 46. FIG. 3 shows a subject S.

The anatomical model 46 is a three-dimensional model in which a standard human body is imitated. The subject S is a person (for example, a patient) who is a target of the ultrasonic examination.

Correct answer positions A, B, C, and D are shown in FIG. 2. The correct answer positions A, B, C, and D are positions on the anatomical model 46 and are examples of the correct answer position Pm. The correct answer positions A, B, C, and D are positions of the correct answer cross sections.

Actual positions α, β, γ, and δ are shown in FIG. 3. The actual positions α, β, γ, and δ are positions on a body surface of the subject S and are positions in the real space. The actual positions α, β, Y, and 8 are examples of the actual position Pr and are positions detected by the position sensor 14.

In the examples shown in FIGS. 2 and 3, the ultrasound probe 12 is actually disposed at each of the actual positions α, β, γ, and δ in sequence, and the scanning with the ultrasonic waves is performed at each actual position in sequence to generate the ultrasound image. For example, the ultrasound probe 12 is disposed at each actual position in the order of the actual positions α, β, γ, and δ, and the scanning with the ultrasonic waves is performed at each actual position. As a result, the ultrasound image is acquired at each of the actual positions α, β, γ, and δ.

In a case in which the transformation matrix M expressed by Expression (2) is a 4×4 matrix, the update unit 40 generates the transformation matrix M based on the position information of the four actual positions α, β, γ, and δ and the position information of the four correct answer positions A, B, C, and D. As described above, the transformation matrix M is expressed by the matrix Pm and the matrix Pr. The matrix Pm is expressed by coordinate values of the four correct answer positions. The matrix Pr is expressed by four coordinate values of the four actual positions.

Specifically, the ultrasound probe 12 is disposed at the actual position α, and the actual position α is detected by the position sensor 14. The update unit 40 inputs the coordinate value of the actual position α into a column of the matrix Pr.

The recognition unit 36 estimates the scanning cross section scanned with the ultrasonic waves in a case in which the ultrasound probe 12 is disposed at the actual position α. That is, the correct answer cross section estimated to match the scanning cross section actually scanned with the ultrasonic waves is specified from the anatomical model 46. The correct answer cross section and the correct answer position Pm are registered in the anatomical model 46, and the update unit 40 specifies the correct answer position Pm of the estimated correct answer cross section from the anatomical model 46. For example, the update unit 40 specifies the correct answer position A as the correct answer position Pm of the estimated correct answer cross section. That is, the correct answer position of the correct answer cross section estimated to match the scanning cross section of the actual position α is the correct answer position A. It can be said that the correct answer position A is a position on the anatomical model 46 that corresponds to the actual position α. The update unit 40 inputs the coordinate value of the correct answer position A to a column of the matrix Pm corresponding to the column to which the coordinate value of the actual position α is input.

The coordinate values of the actual positions β, γ, and δ are also input to the matrix Pr in the same manner as the coordinate value of the actual position α. The coordinate values of the correct answer positions B, C, and D are also input to the matrix Pm in the same manner as the coordinate value of the correct answer position A.

That is, the ultrasound probe 12 is disposed at the actual position β, and the actual position β is detected by the position sensor 14. The update unit 40 inputs the coordinate value of the actual position β into a column of the matrix Pr.

The recognition unit 36 estimates the scanning cross section scanned with the ultrasonic waves in a case in which the ultrasound probe 12 is disposed at the actual position β. The update unit 40 specifies the correct answer position B as the correct answer position Pm of the estimated correct answer cross section. That is, the correct answer position of the correct answer cross section estimated to match the scanning cross section of the actual position β is the correct answer position B. It can be said that the correct answer position B is a position on the anatomical model 46 that corresponds to the actual position β. The update unit 40 inputs the coordinate value of the correct answer position B to a column of the matrix Pm corresponding to the column to which the coordinate value of the actual position β is input.

The correct answer position of the correct answer cross section estimated to match the scanning cross section of the actual position γ is the correct answer position C. The update unit 40 inputs the coordinate value of the actual position γ into a column of the matrix Pr. The update unit 40 inputs the coordinate value of the correct answer position C into a column of the matrix Pm.

The correct answer position of the correct answer cross section estimated to match the scanning cross section of the actual position δ is the correct answer position D. The update unit 40 inputs the coordinate value of the actual position δ into a column of the matrix Pr. The update unit 40 inputs the coordinate value of the correct answer position D into a column of the matrix Pm.

The update unit 40 generates the transformation matrix M based on the matrix Pr consisting of the coordinate values of the actual positions α, β, γ, and δ and the matrix Pm consisting of the coordinate values of the correct answer positions A, B, C, and D in accordance with Expression (2). As a result, a 4×4 transformation matrix M is generated.

Thereafter, in a case in which the scanning with the ultrasonic waves is performed at a new actual position to generate an ultrasound image, the update unit 40 updates the transformation matrix M by inputting a coordinate value of the new actual position to the matrix Pr and inputting a coordinate value of a correct answer position corresponding to the new actual position to the matrix Pm.

For example, the update unit 40 replaces the coordinate value input in the column of the earliest input order in the matrix Pr with the coordinate value of the new actual position. Similarly, the update unit 40 replaces the coordinate value input in the column of the earliest input order in the matrix Pm with the coordinate value of the correct answer position corresponding to the new actual position. For example, in a case in which the scanning with the ultrasonic waves is performed at the actual position δ and then the ultrasound probe 12 is disposed at an actual position ε to generate an ultrasound image, the update unit 40 inputs a coordinate value of the actual position ε to the column of the matrix Pr to which the coordinate value of the actual position α is input, instead of the coordinate value of the actual position α. Similarly, the update unit 40 inputs a coordinate value of the correct answer position corresponding to the actual position ε to the column of the matrix Pm to which the coordinate value of the correct answer position A is input, instead of the coordinate value of the correct answer position A. In this way, the update unit 40 updates the transformation matrix M.

Of course, this update example is merely an example. The update unit 40 may generate the transformation matrix M by inputting a coordinate value of the actual position at which the scanning cross section having a high degree of certainty is acquired to the matrix Pr and inputting a correct answer position corresponding to the actual position to the matrix Pm. In this way, it is possible to generate the transformation matrix M that enables more accurate position transformation. For example, in a case in which the transformation matrix M is a 4×4 matrix, the update unit 40 updates the transformation matrix M by using the top four coordinate values (the coordinate value of the actual position and the coordinate value of the correct answer position) with the highest degrees of certainty, from the first to the fourth. As another example, the update unit 40 may determine an average value of a plurality of transformation matrices M used in the past as the transformation matrix M to be used for the transformation. The update unit 40 may calculate a weighted transformation matrix M by using an average value of the degrees of certainty at the respective positions as a weighting coefficient.

For example, the transformation matrix M is generated by using the first four frames of the ultrasound images immediately after the start of the ultrasonic examination. For example, in a case in which a frame rate is 20, the transformation matrix M is generated in 0.2 seconds. As the examination progresses, the transformation matrix M is updated, and the transformation matrix M that enables more accurate position transformation is generated.

In the example shown in FIG. 3, the actual positions are spaced from each other, but the actual positions may be close to each other. For example, the transformation matrix M may be generated by acquiring the ultrasound images at a plurality of positions while slightly changing the position of the ultrasound probe 12 in the vicinity of the actual position α.

As still another example, the transformation matrix M may be generated by performing the scanning with the ultrasonic waves in a wider region at the beginning of the examination.

In the above-described embodiment, the scanning cross section is estimated and the transformation matrix M is generated, but as another example, the recognition unit 36 may estimate a site scanned with the ultrasonic waves, and the transformation unit 38 may generate the transformation matrix M by using a position of the site. For example, the site is a kidney, a gallbladder, an abdominal aorta, a hepatic vein bifurcation, or the like. For example, the site and a correct answer position of the site are registered in the anatomical model in advance for each site. The transformation unit 38 specifies the correct answer position corresponding to the site estimated by the recognition unit 36 from the anatomical model, and generates the transformation matrix M based on the actual position detected by the position sensor 14 and the correct answer position.

In a case in which the transformation matrix M is generated as described above, the transformation unit 38 transforms the actual position Pr detected by the position sensor 14 into the correct answer position Pm on the anatomical model by using the transformation matrix M in accordance with Expression (1). The display controller 28 displays extraction information corresponding to the correct answer position Pm on the display unit 22.

In a case in which the transformation matrix M is updated by the update unit 40, the transformation unit 38 transforms the actual position Pr into the correct answer position Pm by using the updated transformation matrix M. The display controller 28 displays extraction information corresponding to the correct answer position Pm on the display unit 22.

For example, in a case in which an actual position ξ of the ultrasound probe 12 is detected by the position sensor 14, the transformation unit 38 transforms the actual position ξ into the correct answer position on the anatomical model by using the transformation matrix M. The display controller 28 displays extraction information corresponding to the correct answer position on the display unit 22. In addition, the update unit 40 updates the transformation matrix M by using a coordinate value of the actual position ξ and a coordinate value of the correct answer position corresponding to the actual position ξ.

### EXAMPLES

Hereinafter, examples will be described. Here, as an example, ultrasonic examination targeted for abdominal examination will be described. For example, 25 correct answer cross sections of the abdomen are registered in advance in the anatomical model. Specifically, center coordinates (that is, the correct answer position) of each correct answer cross section, a coordinate range of the correct answer cross section, a name of the correct answer cross section, and an orientation of the viewpoint with respect to the correct answer cross section are registered in advance in the anatomical model. Data of the anatomical model is stored in the storage unit 24 in advance.

For example, an initial value of the transformation matrix M is a unit matrix. At this stage, the transformation using the transformation matrix M is not accurately performed, so that the extraction information is not displayed.

In the examination, usually, first, bird's-eye view scanning is performed. For example, the scanning with the ultrasonic waves is performed at a plurality of positions, whereby an overall image of the examination is confirmed by the user such as the examination technician. The update unit 40 generates the initial transformation matrix M based on the position information acquired during the scanning and the result of the recognition by the recognition unit 36. For example, in a case in which four cross sections are scanned, the update unit 40 generates the initial transformation matrix M, which is a 4×4 matrix. In a case in which a different site is detected by detecting a site (for example, an organ) in a case in which the bird's-eye view scanning is performed to scan a narrow range without switching the scanning cross section, the update unit 40 generates the transformation matrix M by using four positions of the detected site.

In a case in which the transformation matrix M is generated, the transformation unit 38 transforms the actual position Pr detected by the position sensor 14 into the correct answer position Pm by using the transformation matrix M. The display controller 28 displays extraction information corresponding to the correct answer position Pm on the display unit 22.

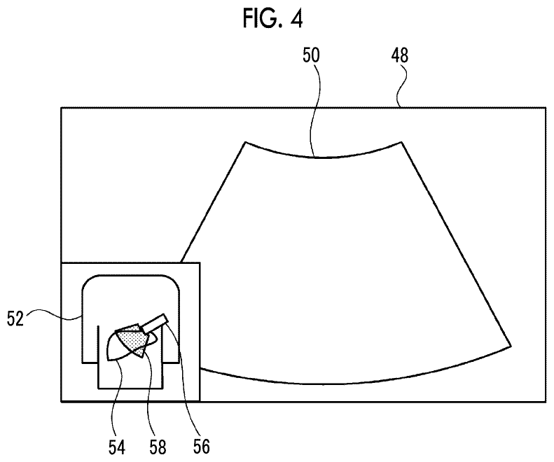

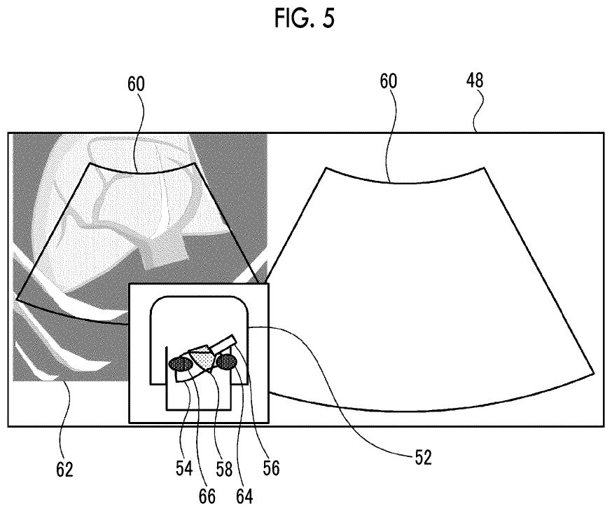

A display example of the extraction information will be described with reference to FIGS. 4 and 5. FIGS. 4 and 5 show display examples of an ultrasound image and a body mark.

A screen 48 shown in FIG. 4 is displayed on the display unit 22. In a case in which an ultrasound image 50 is generated by the scanning with the ultrasonic waves, the display controller 28 displays the ultrasound image 50 on the screen 48. For example, the ultrasound image 50 is a B-mode image generated by scanning a certain scanning cross section. Of course, an image other than the B-mode image may be acquired and displayed as the ultrasound image.

As shown in FIG. 4, the display controller 28 displays a body mark 52 on the screen 48. The body mark 52 is a two-dimensional image or a three-dimensional image in which the subject is imitated. Here, as an example, the body mark 52 is a three-dimensional image (that is, a 3D body mark).

For example, the display controller 28 displays an examination region 54 on the body mark 52. In addition, the display controller 28 displays a probe mark 56 on the body mark 52. The probe mark 56 is an image representing the ultrasound probe 12. The display controller 28 displays the probe mark 56 at a correct answer position corresponding to the current actual position detected by the position sensor 14 on the body mark 52. The current orientation of the ultrasound probe 12 is also detected by the position sensor 14. The display controller 28 expresses the detected orientation of the ultrasound probe 12 in accordance with the tilt of the probe mark 56. In addition, the display controller 28 displays a cross section mark 58 on the body mark 52. The cross section mark 58 is an example of information indicating the current scanning cross section, and is an image representing a correct answer cross section corresponding to the current scanning cross section. In the example shown in FIG. 4, the body mark 52, the examination region 54, the probe mark 56, and the cross section mark 58 correspond to examples of the extraction information.

FIG. 5 shows a display example of a state in which the examination has progressed. An ultrasound image 60 is displayed on the screen 48. As in the example shown in FIG. 4, the body mark 52, the examination region 54, the probe mark 56, and the cross section mark 58 are displayed on the screen 48. As in the example shown in FIG. 4, the probe mark 56 is displayed at a correct answer position corresponding to the current actual position of the ultrasound probe 12. The cross section mark 58 is an image representing a correct answer cross section corresponding to the current scanning cross section.

The display controller 28 displays a 3D model image 62 on the screen 48. The 3D model image 62 is a three-dimensional image representing a site to be examined. The display controller 28 superimposes and displays the ultrasound image 60 on the 3D model image 62.

In addition, the display controller 28 displays cross section marks 64 and 66 on the body mark 52. The cross section marks 64 and 66 are examples of information indicating the non-scanned scanning cross section, and is an image representing a correct answer cross section corresponding to the non-scanned scanning cross section. As described above, the correct answer cross section (for example, 25 correct answer cross sections) to be scanned with the ultrasonic waves is registered in advance in the anatomical model. It is presumed that the scanning cross section recognized by the recognition unit 36 is a cross section scanned with ultrasonic waves. In other words, the non-scanned scanning cross section is a cross section that is not recognized by the recognition unit 36 among the plurality of correct answer cross sections registered in advance in the anatomical model. The display controller 28 displays a cross section mark representing the scanning cross section that is not recognized by the recognition unit 36 on the screen 48 as a cross section mark representing the non-scanned scanning cross section.

The display controller 28 displays the cross section mark 58 representing the current scanning cross section and the cross section marks 64 and 66 representing the non-scanned scanning cross section in different display aspects. For example, the display controller 28 displays the cross section mark 58 and the cross section marks 64 and 66 in different colors. Specifically, the display controller 28 displays the cross section mark 58 in blue and displays the cross section marks 64 and 66 in red. Of course, this display aspect is merely an example, and each cross section mark may be displayed in a different color.

For example, the non-scanned scanning cross section is spatially pre-reset, and a position to be scanned with the ultrasonic waves is set to a displayed state. A position that is not in the displayed state is visualized by processing such as rendering. For example, as the cross section marks 64 and 66, the non-scanned scanning cross section is displayed.

In a case in which the position that is not in the displayed state remains at the end of the examination, the display controller 28 may display a message indicating that the non-scanned scanning cross section exists on the display unit 22.

As another display example, the display controller 28 may display a scanned region and a non-scanned region on the body mark 52. For example, the display controller 28 displays the scanned region and the non-scanned region on the body mark 52 in different display colors. Each region has a shape of an organ. As a result, it is possible to provide the user such as the examination technician with information that is easier to perceive.

In a case in which the body position of the subject is changed, the display controller 28 may display information before the body position is changed and information after the body position is changed in a distinguishable manner on the display unit 22. The information before the body position is changed is information indicating the non-scanned scanning cross section before the body position is changed and information indicating the scanned scanning cross section before the body position is changed. The information after the body position is changed is information indicating the non-scanned scanning cross section after the body position is changed and information indicating the scanned scanning cross section after the body position is changed. For example, the display controller 28 makes the display color different between the information before the body position is changed and the information after the body position is changed. The display controller 28 may switch between display and non-display of the information before the body position is changed in response to a switching instruction from the user. The user can issue an instruction to switch between the display and the non-display by operating the operation unit 32. The display controller 28 may display the information before the body position is changed and the information after the body position is changed in the same color.

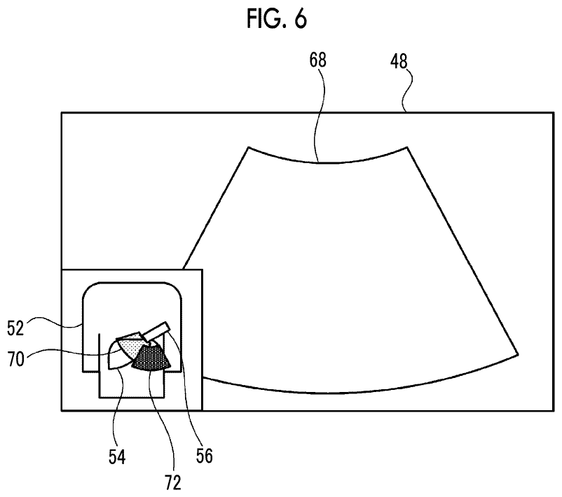

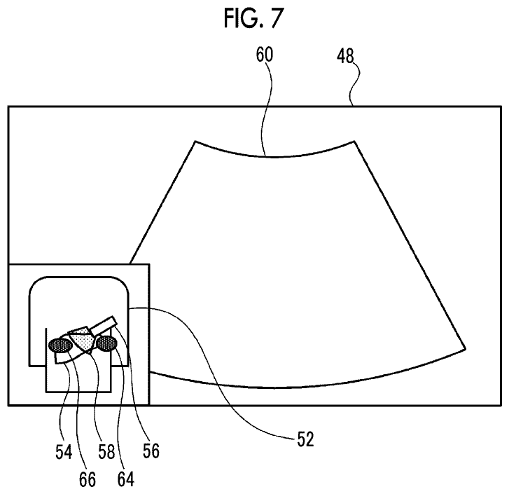

Another display example will be described with reference to FIG. 6. FIG. 6 shows a display example of an ultrasound image and a body mark.

An ultrasound image 68 and the body mark 52 are displayed on the screen 48. In addition, the examination region 54 and the probe mark 56 are displayed on the body mark 52. The probe mark 56 represents a current position and an orientation of the ultrasound probe 12. In addition, cross section marks 70 and 72 are displayed on the body mark 52. The cross section mark 70 is an image representing a current scanning cross section. The cross section mark 72 is an image representing a scanned scanning cross section. For example, the probe mark 56 is displayed in yellow, the cross section mark 70 is displayed in blue, and the cross section mark 72 is displayed in green. By referring to these images, the user can confirm the current position of the ultrasound probe 12, the current scanning cross section, and the scanned scanning cross section. In addition to the color, the probe mark and the cross section mark may be displayed in a distinguishable manner based on the brightness, the chroma saturation, the hue, the type of the line, and the like. The same applies to other display examples.

Another display example will be described with reference to FIG. 7. FIG. 7 shows a display example of an ultrasound image and a body mark.

As in the display example shown in FIG. 5, the ultrasound image 60 and the body mark 52 are displayed on the screen 48. In addition, the examination region 54 and the probe mark 56 are displayed on the body mark 52. The probe mark 56 represents a current position and an orientation of the ultrasound probe 12. In addition, cross section marks 58, 64, and 66 are displayed on the body mark 52. The cross section mark 58 is an image representing a current scanning cross section. The cross section marks 64 and 66 are images representing non-scanned scanning cross sections. For example, the cross section marks 64 and 66 are images representing non-scanned cross sections among cross sections to be scanned (for example, a cross section of a liver vein and a cross section immediately below a diaphragm). The probe mark 56 is displayed in yellow, the cross section mark 58 is displayed in blue, and the cross section marks 64 and 66 are displayed in red. By referring to these images, the user can confirm the current position of the ultrasound probe 12, the current scanning cross section, and the non-scanned scanning cross section.

The extraction information may be displayed in accordance with the orientation of the ultrasound probe 12 set in the anatomical model, the orientation of the viewpoint, or the orientation of the correct answer cross section. For example, an image of the anatomical model in a bird's-eye view from a fixed position, an image of the anatomical model viewed from the same viewpoint as the generated ultrasound image, or an image of the anatomical model viewed from the same viewpoint as a standard cross-sectional image may be displayed as the extraction information. In a case in which the user issues a switching instruction, these images may be switched and displayed.

In a case in which the ultrasonic examination is ended, the display controller 28 may display information indicating the non-scanned scanning cross section on the display unit 22. In this way, the user can perceive the site where the scanning is not performed.

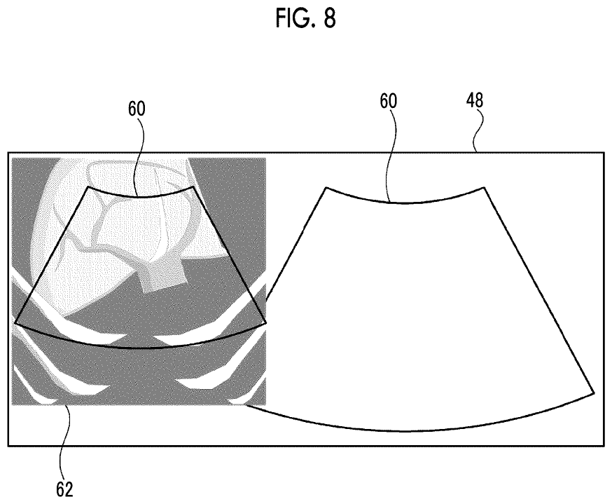

Another display example will be described with reference to FIG. 8. FIG. 8 shows a display example of an ultrasound image. As in the display example shown in FIG. 5, the ultrasound image 60 and the 3D model image 62 are displayed on the screen 48. The ultrasound image 60 is superimposed and displayed on the 3D model image 62. The 3D model image 62 and the ultrasound image 60 superimposed on the 3D model image 62 are displayed as the extraction information.

As the data of the anatomical model, standard three-dimensional ultrasound data, computed tomography (CT) data, or magnetic resonance imaging (MRI) data may be used. As another example, as the data of the anatomical model, past ultrasound data, CT data, or MRI data of the subject (for example, the patient) to be examined may be used.

Hereinafter, each modification example will be described.

### Modification Example 1

The anatomical model may be created in advance for each examination site, and data of the anatomical model of each examination site may be stored in the storage unit 24 in advance. In this case, the correct answer cross section and the correct answer position are registered in the anatomical model in advance for each examination site. That is, the anatomical model for each examination site may be created in advance. The recognition unit 36 specifies the correct answer cross section from the anatomical model for the site that serves as the target of the ultrasonic examination. That is, the recognition unit 36 specifies the correct answer cross section that is estimated to match the scanning cross section actually scanned with the ultrasonic waves, from the anatomical model that serves as the target of the ultrasonic examination. In addition, the initial transformation matrix M may be determined in advance for each examination site, and the initial transformation matrix M for each examination site may be stored in the storage unit 24. In this case, the transformation unit 38 transforms the actual position Pr into the correct answer position Pm by using the transformation matrix M for the site that serves as the target of the ultrasonic examination. The update unit 40 updates the transformation matrix M for the site that serves as the target of the ultrasonic examination by using the correct answer position Pm of the correct answer cross section corresponding to the scanning cross section recognized by the recognition unit 36 and the actual position Pr. The display controller 28 displays the extraction information obtained from the anatomical model for the site that serves as the target of the ultrasonic examination, on the display unit 22.

### Modification Example 2

The anatomical model may be created in advance for each measurement mode, and data of the anatomical model of each measurement mode may be stored in the storage unit 24 in advance. In this case, the correct answer cross section and the correct answer position are registered in the anatomical model in advance for each measurement mode. That is, the anatomical model for each examination mode may be created in advance. The recognition unit 36 specifies the correct answer cross section from the anatomical model for the examination mode being executed. That is, the recognition unit 36 specifies the correct answer cross section that is estimated to match the scanning cross section actually scanned with the ultrasonic waves, from the anatomical model for the examination mode being executed. In addition, the initial transformation matrix M may be determined in advance for each examination mode, and the initial transformation matrix M for each examination mode may be stored in the storage unit 24. In this case, the transformation unit 38 transforms the actual position Pr into the correct answer position Pm by using the transformation matrix M for the examination mode being executed. The update unit 40 updates the transformation matrix M for the examination mode being executed by using the correct answer position Pm of the correct answer cross section corresponding to the scanning cross section recognized by the recognition unit 36 and the actual position Pr. The display controller 28 displays the extraction information obtained from the anatomical model for the examination mode being executed, on the display unit 22.

### Modification Example 3

The anatomical model may be created in advance for at least one of the body shape or the body position of the subject. That is, the anatomical model may be created in advance for each body shape of the subject, the anatomical model may be created in advance for each body position of the subject during the examination, or the anatomical model may be created in advance for each combination of the body shape and the body position.

The initial transformation matrix M may be determined in advance for at least one of the body shape or the body position of the subject. That is, the initial transformation matrix M may be determined in advance for each body shape of the subject, and the initial transformation matrix M for each body shape may be stored in the storage unit 24 in advance. The initial transformation matrix M may be determined for each body position of the subject, and the initial transformation matrix M for each body position may be stored in the storage unit 24 in advance. The initial transformation matrix M may be determined in advance for each combination of the body shape and the body position of the subject, and the initial transformation matrix M for each combination of the body shape and the body position may be stored in the storage unit 24 in advance.

For example, an anatomical model for a fat body shape, an anatomical model for a standard body shape, and an anatomical model for a thin body shape are created in advance. A depth or a position from the body surface to the examination site (for example, the organ) may vary depending on the body shape of the subject. Therefore, the correct answer cross section or the correct answer position may vary depending on the body shape of the subject. In order to cope with this, an anatomical model for each body shape is created in advance. The correct answer cross section and the correct answer position are registered in the anatomical model in advance for each body shape. That is, the anatomical model for each body shape is created in advance, and data of the anatomical model of each body shape is stored in the storage unit 24 in advance. The recognition unit 36 specifies the correct answer cross section from the anatomical model for the body shape of the subject. That is, the recognition unit 36 specifies the correct answer cross section that is estimated to match the scanning cross section actually scanned with the ultrasonic waves, from the anatomical model for the body shape of the subject. In addition, the initial transformation matrix M may be determined in advance for each body shape, and the initial transformation matrix M for each body shape may be stored in the storage unit 24. In this case, the transformation unit 38 transforms the actual position Pr into the correct answer position Pm by using the transformation matrix M for the body shape of the subject. The update unit 40 updates the transformation matrix M for the body shape of the subject using the correct answer position Pm of the correct answer cross section corresponding to the scanning cross section recognized by the recognition unit 36 and the actual position Pr. The display controller 28 displays the extraction information obtained from the anatomical model for the body shape of the subject, on the display unit 22.

For example, an anatomical model for a supine position, an anatomical model for a lateral position, and an anatomical model for a sitting position are created in advance. A depth or a position from the body surface examination site may vary depending on the body position of the subject. Therefore, the correct answer cross section or the correct answer position may vary depending on the body position of the subject. In order to cope with this, an anatomical model for each body position is created in advance. The correct answer cross section and the correct answer position are registered in the anatomical model in advance for each body position. That is, the anatomical model for each body position is created in advance, and data of the anatomical model of each body position is stored in the storage unit 24 in advance. The recognition unit 36 specifies the correct answer cross section from the anatomical model for the body position of the subject. That is, the recognition unit 36 specifies the correct answer cross section that is estimated to match the scanning cross section actually scanned with the ultrasonic waves, from the anatomical model for the body position of the subject. In addition, the initial transformation matrix M may be determined in advance for each body position, and the initial transformation matrix M for each body position may be stored in the storage unit 24. In this case, the transformation unit 38 transforms the actual position Pr into the correct answer position Pm by using the transformation matrix M for the body position of the subject. The update unit 40 updates the transformation matrix M for the body position of the subject using the correct answer position Pm of the correct answer cross section corresponding to the scanning cross section recognized by the recognition unit 36 and the actual position Pr. The display controller 28 displays the extraction information obtained from the anatomical model for the body position of the subject, on the display unit 22.

Similarly, in a case in which the anatomical model is created for each combination of the body shape and the body position, the position is transformed, the transformation matrix M is updated, and the extraction information is displayed by using the anatomical model corresponding to the combination of the body shape and the body position of the subject.

Here, the processing performed by the setting unit 42 and the estimation unit 44 will be described in detail.

The setting unit 42 sets an anatomical model corresponding to at least one of the body shape or the body position of the subject that serves as the target of the ultrasonic examination, as the anatomical model to be used for the transformation. The recognition unit 36 specifies the correct answer cross section that is estimated to match the scanning cross section actually scanned with the ultrasonic waves, from the anatomical model set by the setting unit 42. The transformation unit 38 transforms the actual position Pr into the correct answer position Pm by the transformation matrix M defined by using the anatomical model set by the setting unit 42. The update unit 40 updates the transformation matrix M by using the anatomical model set by the setting unit 42. The display controller 28 displays the extraction information obtained from the anatomical model set by the setting unit 42 on the display unit 22.

For example, at least one of the body shape or the body position of the subject is designated by the user. The setting unit 42 sets an anatomical model corresponding to the at least one designated by the user as the anatomical model to be used for the transformation. For example, the user determines the body shape and the body position of the subject that serves as the target of the ultrasonic examination, and designates at least one of the body shape or the body position of the subject by operating the operation unit 32.

As another example, the setting unit 42 may estimate at least one of the body shape or the body position of the subject based on an image (for example, a video image or a still image) generated by imaging the subject that serves as the target of the ultrasonic examination with a camera, and set an anatomical model corresponding to a result of the estimation, as the anatomical model to be used for the transformation. As a technique of detecting the body shape and the body position of the subject from the image, a known technique is used. For example, the setting unit 42 estimates the body shape and the body position of the subject represented in the image by using the image recognition technique. In addition, in a case in which the body position of the subject changes during the examination, the setting unit 42 sets an anatomical model corresponding to the body position after the change as the anatomical model to be used for the transformation. As a technique of estimating the body shape and the body position, AI, machine learning, pattern matching, or the like may be used.

Further, as another example, the setting unit 42 may receive subject identification information for identifying the subject, and set an anatomical model corresponding to a body shape indicated by body shape information linked to the subject identification information, as the anatomical model to be used for the transformation.

For example, before the ultrasonic examination is executed, the subject identification information (for example, a patient ID) of the subject that serves as the target of the ultrasonic examination is input to the ultrasound diagnostic apparatus 10. The user may input the subject identification information to the ultrasound diagnostic apparatus 10 by operating the operation unit 32. In a case in which a tag on which the subject identification information is described is attached to a wrist or the like of the subject, the subject identification information may be input to the ultrasound diagnostic apparatus 10 by reading the subject identification information described in the tag. For example, the encoded subject identification information (for example, a barcode or a two-dimensional code) is described in the tag, and the encoded subject identification information is read. The subject identification information may be input to the ultrasound diagnostic apparatus 10 from an external device such as a server, via a communication path such as a network.

The body shape information indicating the body shape of the subject is linked to the subject identification information. The body shape information may be included in the subject identification information, or may be linked to the subject identification information and stored in an external device such as a server. In a case in which the body shape information is stored in the external device and in a case in which the input subject identification information is received, the setting unit 42 acquires the body shape information linked to the input subject identification information and stored in the external device. The setting unit 42 sets an anatomical model corresponding to the body shape indicated by the body shape information as the anatomical model to be used for the transformation.

The estimation unit 44 estimates at least one of the body shape or the body position of the subject based on the position and the posture of the ultrasound probe 12. The position and the posture of the ultrasound probe 12 are detected by the position sensor 14. The setting unit 42 sets an anatomical model corresponding to a result of the estimation by the estimation unit 44 as the anatomical model to be used for the transformation.

For example, in a case in which the subject is a subject having a fat body shape, the examination may be performed by pressing the ultrasound probe 12 more strongly against the body surface of the subject than in a case of a subject having a thin body shape. As a result, the ultrasound probe 12 is pushed further into the body than in a case of examining a subject having a thin body shape. Accordingly, even in a case in which the same ultrasonic examination is executed, the actual position Pr of the ultrasound probe 12 varies depending on the body shape of the subject. Therefore, the body shape of the subject can be estimated based on the actual position Pr of the ultrasound probe 12.

In addition, a position or an orientation of the ultrasound probe 12 on the body surface of the subject varies depending on a body position such as a supine position, a lateral position, or a sitting position. Therefore, the body position of the subject can be estimated based on the actual position Pr of the ultrasound probe 12.

For example, the user sequentially disposes the ultrasound probe 12 at reference points (for example, a head, an abdomen, a back, a toe, and a fingertip) of the subject, and the position sensor 14 detects the position and the posture of the ultrasound probe 12 at each reference point. The estimation unit 44 estimates a size, a thickness, and a posture of the subject based on the position and the posture of the ultrasound probe 12 at each reference point. The size, the thickness, and the posture of the subject estimated from the position and the posture of the ultrasound probe 12 at each reference position are different depending on the body shape and the body position of the subject. Therefore, the body shape or the body position of the subject can be estimated based on the position and the posture of the ultrasound probe 12 at each reference point.

The estimation unit 44 may estimate at least one of the body shape or the body position of the subject based on the ultrasound image. For example, the estimation unit 44 estimates at least one of the body shape or the body position of the subject based on a shape, a position, and the number of structures such as sites represented in the ultrasound image.

The shape or the position of the structure such as the site may be different depending on the body shape of the subject. Therefore, the body shape of the subject can be estimated based on the shape or the position of the structure represented in the ultrasound image. In addition, the shape or the position of the structure such as the site may be different depending on the body position of the subject. Therefore, the body position of the subject can be estimated according to the shape or the position of the structure represented in the ultrasound image.

For example, a reference point (for example, an abdominal organ) of the subject is scanned with the ultrasonic waves, thereby generating an ultrasound image representing the reference point. For example, an ultrasound image representing the aorta, the spine, the fat layer, and the like is generated. The estimation unit 44 measures a position (for example, a depth from a body surface) of the organ represented in the ultrasound image, the number of the organs, and a size (for example, a thickness) of the organ by applying the recognition processing to the ultrasound image. The estimation unit 44 estimates the body shape or the body position of the subject based on the measurement result. For example, the organ of the subject having a fat body shape is located at a deeper position from the body surface than an organ of the subject having a slim body shape. Accordingly, the body shape of the subject can be estimated based on the position (for example, the depth from the body surface) of the organ represented in the ultrasound image. In addition, the fat layer is thinner in the lying position than in the sitting position. Therefore, the body position of the subject can be estimated based on the thickness of the fat layer represented in the ultrasound image.

In a case in which the body position of the subject is changed, the update unit 40 may create a new transformation matrix M corresponding to the body position after the change. In a case in which the change in the body position of the subject is detected, the update unit 40 resets the transformation matrix M that has been updated so far, and creates a new transformation matrix M based on the actual position Pr detected after the body position is changed and the correct answer position Pm corresponding to the actual position Pr. Thereafter, the update unit 40 continues to update the newly created transformation matrix M. For example, the estimation unit 44 can detect the change in the body position of the subject based on the image (for example, a video image or a still image) generated by imaging the subject with the camera. As another example, in a case in which the actual position Pr detected by the position sensor 14 and the scanning cross section do not match the current transformation matrix M, the estimation unit 44 may determine that the body position of the subject has changed.

The storage controller 30 may store the extraction information and the information indicating the body position of the subject in the storage unit 24 in association with each other for each body position of the subject. For example, in a case in which the body position of the subject is changed, the storage controller 30 stores the information indicating the body position before the change and the extraction information before the body position is changed in the storage unit 24 in association with each other. In a case in which the user designates the body position before the change by operating the operation unit 32, the display controller 28 displays the extraction information associated with the information indicating the body position before the change on the display unit 22. For example, the display controller 28 displays the extraction information before the body position is changed on the display unit 22 as the past extraction information. The display controller 28 may display the extraction information after the body position is changed (for example, the extraction information corresponding to the current body position) and the extraction information before the body position is changed (for example, the past extraction information) side by side on the display unit 22. In this case, the display controller 28 displays the extraction information after the body position is changed and the extraction information before the body position is changed in a distinguishable manner on the display unit 22. For example, the display controller 28 displays each extraction information on the display unit 22 by changing the brightness, the chroma saturation, the hue, the type of the line, or the like between the extraction information corresponding to the current body position and the past extraction information. The display controller 28 may display the scanned scanning cross section and the non-scanned scanning cross section in a distinguishable manner on the display unit 22. In addition, in a case in which the user issues an instruction to switch the extraction information by operating the operation unit 32, the display controller 28 may switch the extraction information and display the switched extraction information on the display unit 22 in response to the instruction. For example, the display may be switched between the extraction information corresponding to the current body position and the past extraction information.

The image generation unit 18, the display processing unit 20, the analysis unit 34, and the controller 26 can be implemented by using, for example, hardware resources such as a processor and an electronic circuit, and a device such as a memory may be used in the realization as necessary. In addition, the image generation unit 18, the display processing unit 20, the analysis unit 34, and the controller 26 may be implemented by, for example, a computer. That is, all or a part of the image generation unit 18, the display processing unit 20, the analysis unit 34, and the controller 26 may be implemented by cooperation between hardware resources such as a central processing unit (CPU) or a memory included in a computer and software (program) that defines the operation of the CPU or the like. The program is stored in the storage device of the ultrasound diagnostic apparatus 10 or another storage device through a recording medium such as a CD or a DVD or through a communication path such as a network. As another example, the image generation unit 18, the display processing unit 20, the analysis unit 34, and the controller 26 may be implemented by a digital signal processor (DSP), an application specific integrated circuit (ASIC), a field programmable gate array (FPGA), or the like. Of course, a graphics processing unit (GPU) and the like may be used. The image generation unit 18, the display processing unit 20, the analysis unit 34, and the controller 26 may be implemented by a single device or may be implemented by a plurality of devices.

The functions of the image generation unit 18, the display processing unit 20, the analysis unit 34, and the controller 26 may be executed by an apparatus (for example, a personal computer or a server) other than the ultrasound diagnostic apparatus 10.

What is claimed is:

1. An ultrasound diagnostic apparatus comprising:
an acquisition unit that acquires position information of an ultrasound probe;
a recognition unit that recognizes, based on an ultrasound image acquired by scanning with an ultrasonic wave using the ultrasound probe, a scanning cross section scanned with the ultrasonic wave;
a transformation unit that uses an anatomical model in which a correct answer cross section on which ultrasonic examination is to be performed and a correct answer position of the correct answer cross section are registered, to transform a position indicated by the position information acquired by the acquisition unit into the correct answer position associated with the anatomical model according to a transformation rule;
a display controller that displays extraction information related to the ultrasonic examination, which corresponds to the correct answer position transformed by the transformation unit, on a display; and
an update unit that updates the transformation rule by using the correct answer position of the correct answer cross section corresponding to the scanning cross section recognized by the recognition unit and the position indicated by the position information acquired by the acquisition unit.

2. The ultrasound diagnostic apparatus according to claim 1,
wherein the transformation according to the transformation rule is transformation using a transformation matrix, and
the update unit updates the transformation matrix by using the position indicated by the position information acquired by the acquisition unit and the correct answer position of the correct answer cross section.

3. The ultrasound diagnostic apparatus according to claim 1,
wherein the correct answer cross section and the correct answer position are registered in the anatomical model for each examination site.

4. The ultrasound diagnostic apparatus according to claim 1, further comprising:
a setting unit that sets an anatomical model corresponding to at least one of a body shape or a body position of a subject that serves as a target of the ultrasonic examination, as the anatomical model to be used for the transformation.

5. The ultrasound diagnostic apparatus according to claim 4,
wherein at least one of the body shape or the body position of the subject is designated by a user, and
the setting unit sets an anatomical model corresponding to the at least one designated by the user, as the anatomical model to be used for the transformation.

6. The ultrasound diagnostic apparatus according to claim 4,
wherein the setting unit estimates at least one of the body shape or the body position of the subject based on an image generated by imaging the subject with a camera, and sets an anatomical model corresponding to a result of the estimation, as the anatomical model to be used for the transformation.

7. The ultrasound diagnostic apparatus according to claim 4,
wherein the setting unit receives subject identification information for identifying the subject, and sets an anatomical model corresponding to a body shape indicated by body shape information linked to the subject identification information, as the anatomical model to be used for the transformation.

8. The ultrasound diagnostic apparatus according to claim 4, further comprising:
an estimation unit that estimates at least one of the body shape or the body position of the subject based on a position of the ultrasound probe,
wherein the setting unit sets an anatomical model corresponding to a result of the estimation by the estimation unit, as the anatomical model to be used for the transformation.

9. The ultrasound diagnostic apparatus according to claim 4, further comprising:
an estimation unit that estimates at least one of the body shape or the body position of the subject based on the ultrasound image,
wherein the setting unit sets an anatomical model corresponding to a result of the estimation by the estimation unit, as the anatomical model to be used for the transformation.

10. The ultrasound diagnostic apparatus according to claim 4,
wherein, in a case in which a change occurs in the body position of the subject, the update unit creates a new transformation rule corresponding to the changed body position.

11. The ultrasound diagnostic apparatus according to claim 1,
wherein the extraction information includes at least one of information indicating a position of the ultrasound probe, information indicating an orientation of the ultrasound probe, information indicating a current scanning range with an ultrasonic wave, information indicating a past scanning range with an ultrasonic wave, or information indicating a non-scanned range.
